(19) United States
(12) Patent Application Publication
(10) Pub. No.: US 2022/0285457 A1
(43) Pub. Date: Sep. 8, 2022

(54) DISPLAY DEVICE

(71) Applicant: Samsung Display Co., LTD., Yongin-si (KR)

(72) Inventors: Jeong Heon LEE, Hwaseong-si (KR); Si Young CHOI, Asan-si (KR)

(73) Assignee: Samsung Display Co., LTD., Yongin-si (KR)

(21) Appl. No.: 17/523,410

(22) Filed: Nov. 10, 2021

(30) Foreign Application Priority Data
Mar. 2, 2021 (KR) 10-2021-0027354

(51) Int. Cl. H01L 27/32

(52) U.S. Cl. CPC H01L 27/3213; H01L 27/3216; H01L 27/3218; H01L 27/3246

(57) ABSTRACT

A display device includes a first light-emitting element, a second light-emitting element, a third light-emitting element, and a fourth light-emitting element disposed on a substrate, wherein the first light-emitting element, the second light-emitting element, and the third light-emitting element emit visible light, the fourth light-emitting element emits invisible light, and a size of a light emission area of the fourth light-emitting element is smaller than a size of a light emission area of each of the first to third light-emitting elements in a plan view.

### CROSS-REFERENCE TO RELATED APPLICATION(S)

This application claims priority to and the benefit of Korean Patent Application No. 10-2021-0027354 under 35 U.S.C. § 119, filed in the Korean Intellectual Property Office (KIPO) on Mar. 2, 2021, the entire contents of which are incorporated herein by reference.

### BACKGROUND

### 1. Technical Field

The disclosure relates to a display device.

### 2. Description of the Related Art

A display device is a device that displays a screen, and includes a liquid crystal display (LCD), an organic light emitting diode (OLED), and the like. The display device is used in various electronic devices such as a mobile phone, a navigation device, a digital camera, an electronic book, a portable game machine, and various terminals.

The organic light emitting diode display includes two electrodes and an organic emission layer disposed between the two electrodes, and electrons injected from one electrode and holes injected from the other electrode are combined in the organic emission layer such that excitons are formed. When the excitons change from an excited state to a ground state, energy is emitted and they emit light.

The organic light emitting diode display includes pixels including an organic light emitting diode that is a self-light emitting element, and each pixel includes transistors and at least one capacitor for driving the organic light emitting diode. Pixels may include pixels displaying different colors, and may include, for example, red pixels, green pixels, and blue pixels.

The above information disclosed in this Background section is only for enhancement of understanding of the background of the described technology, and therefore it may contain information that does not form the prior art that is already known in this country to a person of ordinary skill in the art.

### SUMMARY

An embodiment is to provide a display device having an additional function other than a function of displaying an image by further including a pixel that emits visible light.

In addition, it is to provide a display device that may minimize (or reduce) an effect of the additional pixel on other pixels.

A display device according to an embodiment includes a first light-emitting element, a second light-emitting element, a third light-emitting element, and a fourth light-emitting element disposed on a substrate. The first light-emitting element, the second light-emitting element, and the third light-emitting element may emit visible light, the fourth light-emitting element may emit invisible light, and a size of a light emission area of the fourth light-emitting element may be smaller than a size of a light emission area of each of the first to third light-emitting elements in a plan view.

The fourth light-emitting element may emit ultraviolet light or infrared light. The display device according to an embodiment may further include a first pixel in which the first light-emitting element is disposed, a second pixel in which the second light-emitting element is disposed, a third pixel in which the third light-emitting element is disposed, and a fourth pixel in which the fourth light-emitting element is disposed, wherein the first pixel, the second pixel, the third pixel, and the fourth pixel may form a pixel group.

The first pixel, the second pixel, the third pixel, and the fourth pixel may have a rectangle shape, and the second pixel and the fourth pixel may be disposed between the first pixel and the third pixel.

A width of each of the first pixel, the second pixel, the third pixel, and the fourth pixel in a row direction may be equal to each other, and a length of the fourth pixel in a column direction may be shorter than a length of each of the first pixel, the second pixel, and the third pixel in a column direction.

The display device according to an embodiment may further include a first pixel in which the first light-emitting element is disposed, a second pixel in which the second light-emitting element is disposed, a third pixel in which the third light-emitting element is disposed, and a fourth pixel in which the fourth light-emitting element is disposed, wherein one first pixel, two second pixels, one third pixel, and two fourth pixels may form a pixel group.

The pixel group may have a rhombus shape, the first pixel and the third pixel may each have a rhombus shape, and the second pixel and the fourth pixel may each have a rectangle shape.

The first pixel may be disposed on a left part of the pixel group, the third pixel may be disposed on a right part of the pixel group, the second pixel and the fourth pixel may be disposed on an upper part of the pixel group, and the second pixel and the fourth pixel may be disposed on a lower part of the pixel group.

The first light-emitting element may include a first pixel electrode disposed on the substrate; and a first emission layer disposed on the first pixel electrode and including an organic material emitting a first color light, the second light-emitting element may include a second pixel electrode disposed on the substrate; and a second emission layer disposed on the second pixel electrode and including an organic material emitting a second color light, the third light-emitting element may include a third pixel electrode disposed on the substrate; and a third emission layer disposed on the third pixel electrode and including an organic material emitting a third color light, and the fourth light-emitting element may include a fourth pixel electrode disposed on the substrate; and a fourth emission layer disposed on the fourth pixel electrode and including an organic material emitting ultraviolet light.

A distance between the fourth emission layer and the first to third emission layers may be disposed farther than a distance between the first to third emission layers.

The display device according to an embodiment may further include a bank including a first pixel opening overlapping the first pixel electrode, a second pixel opening overlapping the second pixel electrode, a third pixel opening overlapping the third pixel electrode, and a fourth pixel opening overlapping the fourth pixel electrode. The first emission layer may be disposed in the first pixel opening, the second emission layer may be disposed in the second pixel opening, the third emission layer may be disposed in the third pixel opening, and the fourth emission layer may be disposed in the fourth pixel opening.

A width of the bank surrounding the fourth emission layer may be greater than a width of the bank disposed between the first emission layer and the second emission layer, and a width of the bank surrounding the fourth emission layer may be greater than a width of the bank disposed between the second emission layer and the third emission layer.

A width of the fourth pixel opening may be narrower than a width of each of the first to third pixel openings.

A width of the fourth emission layer may be narrower than a width of each of the first to third emission layers.

A thickness of the fourth emission layer may be thinner than a thickness of each of the first to third emission layers.

The display device according to an embodiment may further include a common electrode disposed on the first emission layer, the second emission layer, the third emission layer, and the fourth emission layer and extending from the first emission layer to the fourth emission layer.

The substrate may include a first display area and a second display area, and the fourth light-emitting element may not be disposed in the first display area and may be disposed in the second display area.

The first light-emitting element, the second light-emitting element, and the third light-emitting element may be disposed in the first display area, and the first light-emitting element, the second light-emitting element, the third light-emitting element, and the fourth light-emitting element may be disposed in the second display area.

The substrate may include second display areas, and second display areas may be disposed to be spaced apart by a constant interval.

The substrate may include second display areas, and second display areas may be disposed in a row direction and a column direction.

According to the embodiment, the display device further includes a pixel that emits invisible light, so that it may have other additional functions such as sterilization in addition to the function of displaying an image.

In addition, it is possible to minimize the effect of the added pixel on other pixels.

### BRIEF DESCRIPTION OF THE DRAWINGS

An additional appreciation according to the embodiments of the disclosure will become more apparent by describing in detail the embodiments thereof with reference to the accompanying drawings, wherein:

FIG. 1 is a schematic plan view of a display device according to an embodiment.

FIG. 2 is a schematic plan view showing a part of a display device according to an embodiment.

FIG. 3 is a schematic cross-sectional view taken along line of FIG. 2.

FIG. 4 is a schematic plan view of a display device according to an embodiment.

FIG. 5 is a schematic plan view of a display device according to an embodiment.

FIG. 6 is a schematic cross-sectional view of a display device according to an embodiment.

FIG. 7 is a schematic cross-sectional view of a display device according to an embodiment.

FIG. 8 is a schematic cross-sectional view of a display device according to an embodiment.

FIG. 9 to FIG. 11 are schematic plan views of a display device according to an embodiment.

### DETAILED DESCRIPTION OF THE EMBODIMENTS

The disclosure will be described more fully hereinafter with reference to the accompanying drawings, in which embodiments of the disclosure are shown. As those skilled in the art would realize, the described embodiments may be modified in various different ways, all without departing from the spirit or scope of the disclosure.

Descriptions of parts not related to the disclosure are omitted, and like reference numerals designate like elements throughout the specification.

Furthermore, as the size and thickness of the respective structural components shown in the drawings are arbitrarily illustrated for explanatory convenience, the disclosure is not necessarily limited to as illustrated. In the drawings, the thickness of layers, films, panels, regions, etc., may be exaggerated for clarity. In the drawings, for better understanding and ease of description, the thicknesses of some layers and areas may be exaggerated.

It will be understood that when an element such as a layer, film, region, or substrate is referred to as being “on” another element, it can be directly on the other element or intervening elements may also be present. In contrast, when an element is referred to as being “directly on” another element, there are no intervening elements present. In addition, the word “on” means positioning on or below the object portion, but does not essentially mean positioning on the upper side of the object portion based on a gravity direction.

In addition, unless explicitly described to the contrary, the word “comprise,” and variations such as “comprises” or “comprising,” will be understood to imply the inclusion of stated elements but not the exclusion of any other elements.

Further, in the specification, the phrase “in a plan view” means when an object portion is viewed from above, and the phrase “in a cross-section” means when a cross-section taken by vertically cutting an object portion is viewed from the side.

The terms “about” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

It will be understood that the terms “contact,” “connected to,” and “coupled to” may include a physical and/or electrical contact, connection or coupling.

The phrase “at least one of” is intended to include the meaning of “at least one selected from the group of” for the purpose of its meaning and interpretation. For example, “at least one of A and B” may be understood to mean “A, B, or A and B.”

Unless otherwise defined or implied herein, all terms (including technical and scientific terms) used herein have the same meaning as commonly understood by those skilled in the art to which this disclosure pertains. It will be further understood that terms, such as those defined in commonly used dictionaries, should be interpreted as having a meaning that is consistent with their meaning in the context of the relevant art and the disclosure, and should not be interpreted in an ideal or excessively formal sense unless clearly so defined herein.

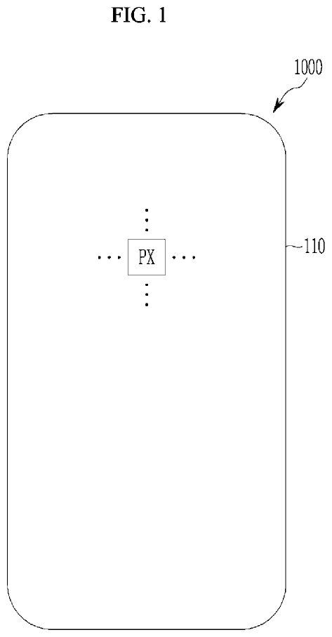

First, a display device according to an embodiment is described with reference to FIG. 1.

FIG. 1 is a schematic plan view showing a display device according to an embodiment.

As shown in FIG. 1, a display device 1000 according to an embodiment includes a substrate 110 and pixels PX disposed on the substrate 110.

The substrate 110 may be formed of a rigid material or a flexible material.

The substrate 110 may be changed in various forms. The substrate 110 may be flexible, stretchable, foldable, bendable, or rollable.

The substrate 110 may be formed as a rectangle including relatively long sides and short sides, and a corner portion may be formed in a shape having a curved surface that is chamfered.

However, the shape of the substrate 110 is only an example and may be changed to various shapes.

The substrate 110 may include a display area and a peripheral area, and for example, the peripheral area may be formed with a shape surrounding the display area.

Pixels PX may be disposed in the display area of the substrate 110. Pixels PX may be disposed in a matrix, and at least some of pixels PX may receive image signals, thereby displaying images.

In this case, the arrangement of pixels PX may be variously changed. Although not shown, the display device according to an embodiment may further include signal lines. The signal lines may include scan lines, light emission control lines, data lines, driving voltage lines, etc. These signal lines may transmit a scan signal, a light emission control signal, a data signal, and a driving voltage, respectively. Signal lines may be disposed to be crossed in a row direction or a column direction. Also, each pixel PX may include transistors, a capacitor, and at least one light emitting diode (LED), which are connected to signal lines. For example, the display device according to an embodiment may be formed as an organic light emitting diode display. However, the kind of the display device is not limited thereto, and it may be formed as display devices of various kinds. For example, the display device may be a liquid crystal display, a quantum dot display device, a micro LED display device, etc.

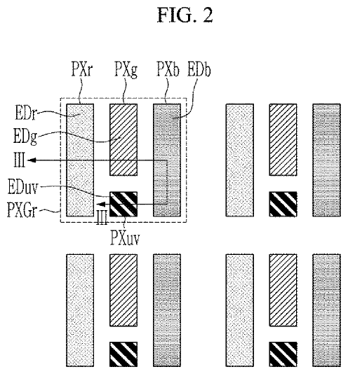

Next, pixels of the display device according to an embodiment are described with reference to FIG. 2.

FIG. 2 is a schematic plan view showing some of a display device according to an embodiment.

As shown in FIG. 2, the display device according to an embodiment includes pixels PXr, PXg, PXb, and PXuv. Pixels PXr, PXg, PXb, and PXuv may include a first pixel PXr, a second pixel PXg, a third pixel PXb, and a fourth pixel PXuv. Each of light-emitting elements EDr, EDg, EDb, and EDuv may be disposed in each of the pixels PXr, PXg, PXb, and PXuv, respectively. The first light-emitting element EDr may be positioned in the first pixel PXr, the second light-emitting element EDg may be positioned in the second pixel PXg, the third light-emitting element EDb may be positioned in the third pixel PXb, and the fourth light-emitting element EDuv may be positioned in the fourth pixel PXuv. The first to fourth light-emitting elements EDr, EDg, EDb, and EDuv may emit light of different wavelengths from each other.

The first light-emitting element EDr, the second light-emitting element EDg, and the third light-emitting element EDb may emit visible light. A wavelength the visible light may be in a range of about 380 nm to about 700 nm. For example, the first light-emitting element EDr may emit red light, and the first pixel PXr may be a red pixel. The second light-emitting element EDg may emit green light, and the second pixel PXg may be a green pixel. The third light-emitting element EDb may emit blue light, and the third pixel PXb may be a blue pixel. However, this is only one example, and colors of light emitted from the first to third pixels PXr, PXg, and PXb may be variously changed. The image may be disposed through a combination of light emitted from the first to third pixels PXr, PXg, and PXb. The fourth light-emitting element EDuv may emit ultraviolet light. Sterilization may be performed by using ultraviolet light emitted from the fourth pixel PXuv. The surface on which the image is displayed on the display device may be contaminated from the outside. For example, when the display device is applied to a portable phone, the upper surface of the display device may be contaminated in the process of touching the display surface of the portable phone and the display surface touching the face while receiving a call. In the display device according to an embodiment, the upper surface of the contaminated display device can be sterilized by driving the fourth pixel PXuv. It is possible to obtain a sterilization effect by matching various items used in daily life such as masks and baby products to the upper surface of the display device and driving the fourth pixel PXuv to emit ultraviolet light. Even if a separate ultraviolet light sterilization device is not portable when going out, various items may be easily sterilized using the portable telephone.

The first to third pixels PXr, PXg, and PXb may be driven simultaneously. The fourth pixel PXuv may be driven with first to third pixels PXr, PXg, and PXb simultaneously or separately.

For example, the fourth pixel PXuv may be selectively driven. For example, even when executing the sterilization function while the display device displays the image, the first to fourth pixels PXr, PXg, PXb, and PXuv may be simultaneously driven.

At this time, the ultraviolet light from the fourth pixel PXuv can be controlled to be discharged to be harmless to the eye of the human body. The fourth pixel PXuv is not driven during the driving of the first to third pixel PXr, PXg, and PXb when the display device does not execute the sterilization function during the display of the image. The image is not displayed by stopping the driving of the first to third pixels PXr, PXg, and PXb, and the fourth pixel PXuv may be driven. In this case, the fourth pixel PXuv may be driven to optimize the sterilization function. Also, the intensity of the ultraviolet light emitted from the fourth pixel PXuv may be controlled by a button or app of the display device.

The first pixel PXr, the second pixel PXg, the third pixel PXb, and the fourth pixel PXuv may form a pixel group PXGr, and pixel groups may be repeatedly disposed. Pixel groups may be disposed in a matrix form along a row direction and a column direction. In a pixel group, the second pixel PXg and the fourth pixel PXuv may be positioned between the first pixel PXr and the third pixel PXb. However, the arrangement shape of the first to fourth pixels PXr, PXg, PXb, and PXuv is only an example and may be variously changed. For example, the positions of the first pixel PXr and the second pixel PXg may be exchanged and the positions of the first pixel PXr and the third pixel PXb may be exchanged.

The size of the fourth pixel PXuv may be relatively smaller than the size of the first to third pixels PXr, PXg, and PXb in a plan view. Each size of the pixels PXr, PXg, PXb, and PXuv may mean the size of a light emission area of each light-emitting element EDr, EDg, EDb, and EDuv. Accordingly, the size of the light emission area of the fourth light-emitting element EDuv may be smaller than the size of the light emission area of the first to third light-emitting elements EDr, EDg, and EDb in a plan view. For example, the size of the fourth pixel PXuv may be about 1% to about 80% of the size of at least one of the first pixel PXr, the second pixel PXg, and the third pixel PXb. The widths of the row direction of the first pixel PXr, the second pixel PXg, the third pixel PXb, and the fourth pixel PXuv may be substantially the same. The lengths of the column direction of the first pixel PXr, the second pixel PXg, the third pixel PXb, and the fourth pixel PXuv may be different. The length of the column direction of the fourth pixel PXuv may be shorter than the length of the column direction of the first to third pixels PXr, PXg, and PXb.

Since the fourth pixel PXuv does not contribute to display of the image, an aperture ratio of the entire display device may be lowered by the fourth pixel PXuv. In the display device according to an embodiment, the fourth pixel PXuv is formed smaller than the first to third pixels PXr, PXg, and PXb, thereby improving the aperture ratio. Also, the first to third pixels PXr, PXg, and PXb may be affected by ultraviolet light emitted from the fourth pixel PXuv. For example, the organic material layer of the first to third pixels PXr, PXg, and PXb may be damaged by ultraviolet light or its life may be shortened. In the display device according to an embodiment, the fourth pixel PXuv may be formed relatively small, the effect of the ultraviolet light emitted from the fourth pixel PXuv on other pixels may be reduced. The size of the fourth pixel PXuv can be selected as an appropriate size in consideration of the sterilization power of the display device. For example, by appropriately designing the size of the fourth pixel PXuv, the aperture ratio may be improved while securing sufficient sterilization power, and the influence on other pixels may be reduced or minimized.

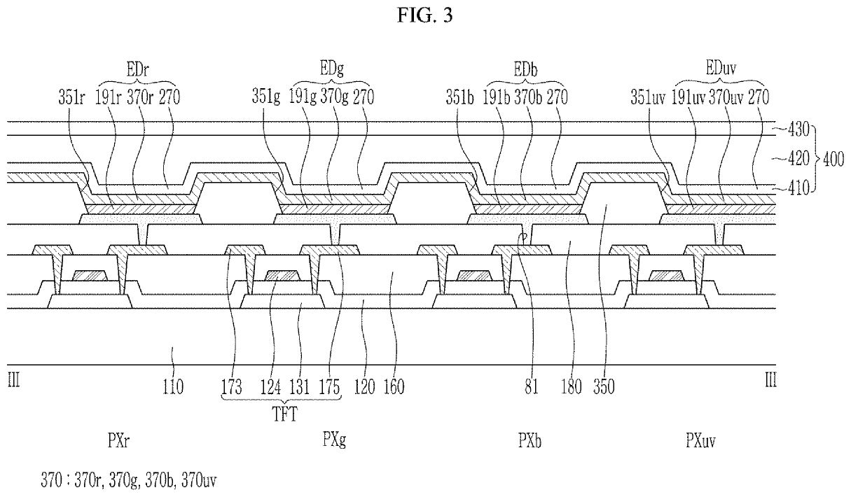

Next, the cross-sectional shape of pixels of the display device according to an embodiment is described in detail with reference to FIG. 3.

FIG. 3 is a schematic cross-sectional view taken along line of FIG. 2.

As shown in FIG. 3, a display device according to an embodiment may include a substrate 110, a transistor TFT including a semiconductor 131, a gate electrode 124, a source electrode 173 and a drain electrode 175, a gate insulating layer 120, an interlayer insulating layer 160, a planarization layer 180, pixel electrodes 191r, 191g, 191b and 191uv, emission layers 370r, 370g, 370b, and 370uv, a partition wall or bank 350, a common electrode 270, and an encapsulation layer 400. Here, the pixel electrodes 191r, 191g, 191b, and 191uv, the emission layers 370r, 370g, 370b, and 370uv and the common electrode 270 may configure (or form) the light-emitting elements EDr, EDg, EDb, and EDuv.

The substrate 110 may include a material that does not bend because of a rigid characteristic such as glass, or may include a flexible material that may be bent such as plastic or polyimide. Although not shown, a lower buffer layer (not shown) or a barrier layer (not shown) may be further positioned on the substrate 110 to flatten the surface of the substrate 110 and block the penetration of impurity elements. In this case, a barrier layer may be positioned on the substrate 110, and a buffer layer may be positioned on the barrier layer. The barrier layer may include an inorganic material, for example may include an inorganic insulating material such as a silicon nitride (SiNₓ), a silicon oxide (SiOₓ), a silicon oxynitride (SiOₓN_{y}), etc. The barrier layer may be a single layer of the material above or a multi-layered structure. The buffer layer may include an inorganic insulating material such as a silicon nitride (SiNₓ), a silicon oxide (SiOₓ), a silicon oxynitride (SiOₓN_{y}), etc. The buffer layer may a single layer of the material or a multi-layered structure.

The semiconductor 131 may be disposed on the substrate 110. The semiconductor 131 may include any of amorphous silicon, polycrystalline silicon, and an oxide semiconductor. For example, the semiconductor 131 may include a low temperature polysilicon (LTPS) or an oxide semiconductor material including at least one of zinc (Zn), indium (In), gallium (Ga), tin (Sn), and a mixture thereof. For example, the semiconductor 131 may include IGZO (Indium-Gallium-Zinc Oxide). The semiconductor 131 may include a channel region, a source region, and a drain region classified according to impurity doping. The source region and drain region may have a conductive characteristic corresponding to a conductor.

The gate insulating layer 120 may cover (or overlap) the semiconductor 131 and the substrate 110. The gate insulating layer 120 may include an inorganic insulating material such as a silicon nitride (SiNₓ), a silicon oxide (SiOₓ), a silicon oxynitride (SiOₓN_{y}), etc. The gate insulating layer 120 may be a single layer of the material or a multi-layered structure.

The gate electrode 124 may be disposed on the gate insulating layer 120. The gate electrode 124 may include a metal or a metal alloy such as copper (Cu), molybdenum (Mo), aluminum (Al), silver (Ag), chromium (Cr), tantalum (Ta), titanium (Ti), and the like. The gate electrode 124 may be composed of (or include) a single layer or a multi-layer. A region of the semiconductor 131 overlapping the gate electrode 124 in a plan view may be a channel region.

The interlayer insulating layer 160 may cover (or overlap) the gate electrode 124 and the gate insulating layer 120. The interlayer insulating layer 160 may include an inorganic insulating material such as a silicon nitride (SiNₓ), a silicon oxide (SiOₓ), a silicon oxynitride (SiOₓN_{y}), and the like. The interlayer insulating layer 160 may be a single layer or a multi-layered structure of the material.

The source electrode 173 and the drain electrode 175 may be disposed on the interlayer insulating layer 160. The source electrode 173 and the drain electrode 175 are connected to the source region and the drain region of the semiconductor 131 by openings formed in the interlayer insulating layer 160 and the gate insulating layer 120, respectively. Accordingly, the aforementioned semiconductor 131, gate electrode 124, source electrode 173, and drain electrode 175 constitute (or form) a transistor (TFT). At least one transistor (TFT) may be positioned in each of the first pixel PXr, the second pixel PXg, the third pixel PXb, and the fourth pixel PXuv. However, the disclosure is not limited thereto, and transistors may be positioned for each pixel PXr, PXg, PXb, and PXuv. Depending on the embodiment, the transistor (TFT) may include only the source region and the drain region of the semiconductor 131 instead of the source electrode 173 and the drain electrode 175.

The source electrode 173 and the drain electrode 175 may include a metal or metal alloys such as aluminum (Al), copper (Cu), silver (Ag), gold (Au), platinum (Pt), palladium (Pd), nickel (Ni), molybdenum (Mo), tungsten (W), titanium (Ti), chromium (Cr), and tantalum (Ta). The source electrode 173 and the drain electrode 175 may be composed of a single layer or a multi-layer. The source electrode 173 and the drain electrode 175 according to an embodiment may be composed of a triple layer including an upper layer, an intermediate layer, and a lower layer, wherein the upper and lower layers may include titanium (Ti), and the intermediate layer may include aluminum (Al).

The planarization layer 180 may be disposed on the source electrode 173 and the drain electrode 175. The planarization layer 180 may cover (or overlap) the source electrode 173, the drain electrode 175, and the interlayer insulating layer 160. The planarization layer 180 is to planarize the surface of the substrate 110 equipped with the transistor (TFT) and may be an organic insulator, and may include at least one material selected from a group consisting of polyimide, polyamide, acryl resin, benzocyclobutene, and phenol resin.

The pixel electrodes 191r, 191g, 191b, and 191uv may be disposed on the planarization layer 180. The pixel electrodes 191r, 191g, 191b, and 191uv are also referred to as anodes, and may be composed of a single layer including a transparent conductive oxide film and a metal material or a multiple layer including these. The transparent conductive oxide layer may include ITO (Indium Tin Oxide), poly-ITO, IZO (Indium Zinc Oxide), IGZO (Indium Gallium Zinc Oxide), ITZO (Indium Tin Zinc Oxide), etc. The metal material may include silver (Ag), molybdenum (Mo), copper (Cu), gold (Au), aluminum (Al), etc. The pixel electrodes 191r, 191g, 191b, and 191uv may include a first pixel electrode 191r positioned in the first pixel PXr, a second pixel electrode 191g positioned in the second pixel PXg, a third pixel electrode 191b positioned in the third pixel PXb, and a fourth pixel electrode 191uv positioned in the fourth pixel PXuv.

The planarization layer 180 may include a via hole 81 (referred to as an opening) exposing the drain electrode 175. The drain electrode 175 and the pixel electrodes 191r, 191g, 191b, and 191uv may be physically and electrically connected through the via hole 81 of the planarization layer 180. Accordingly, the pixel electrodes 191r, 191g, 191b, and 191uv may receive an output current transmitted from the drain electrode 175 to the emission layer 370.

A bank 350 may be positioned on the pixel electrodes 191r, 191g, 191b, and 191uv and the planarization layer 180. The bank 350 is also referred to as a pixel defining layer (PDL) and includes pixel openings 351r, 351g, 351b, and 351uv through which portions of the upper surface of the pixel electrodes 191r, 191g, 191b and 191uv are exposed. The bank 350 may define a formation position of the emission layers 370r, 370g, 370b, and 370uv so that the emission layers 370r, 370g, 370b, and 370uv may be positioned on the exposed portion on the upper surface of the pixel electrodes 191r, 191g, 191b, and 191uv. The pixel openings 351r, 351g, 351b, and 351uv may include a first pixel opening 351r positioned in the first pixel PXr, a second pixel opening 351g positioned in the second pixel PXg, a third pixel opening 351b positioned in the third pixel PXb, and a fourth pixel opening 351uv positioned in the fourth pixel PXuv. The size of each pixel PXr, PXg, PXb, and PXuv in a plan view may be determined by the size of the pixel openings 351r, 351g, 351b, and 351uv. The size of the light emission area of the light-emitting elements EDr, EDg, EDb, and EDuv of each pixel PXr, PXg, PXb, and PXuv may be determined by the size of the pixel openings 351r, 351g, 351b, and 351uv. In the display device according to an embodiment, the size of the fourth pixel opening 351uv may be smaller than that of the first to third pixel openings 351r, 351g, and 351b. For example, the size of the fourth pixel PXuv may be smaller than the size of the first to third pixels PXr, PXg, and PXb. The bank 350 may be an organic insulator including at least one material selected from the group consisting of polyimide, polyamide, acryl resin, benzocyclobutene, and phenol resin. According to an embodiment, the bank 350 may be formed of a black pixel definition layer (BPDL) including a black color pigment.

The emission layers 370r, 370g, 370b, and 370uv may be positioned within the pixel openings 351r, 351g, 351b, and 351uv partitioned by the bank 350. The emission layers 370r, 370g, 370b, and 370uv may include a first emission layer 370r positioned in the first pixel opening 351r, a second emission layer 370g positioned in the second pixel opening 351g, a third emission layer 370b positioned in the third pixel opening 351b, and a fourth emission layer 370uv positioned in the fourth pixel opening 351uv. The first emission layer 370r, the second emission layer 370g, and the third emission layer 370b may include an organic material that emits visible light. For example, the first emission layer 370r may include an organic material that emits red light, the second emission layer 370g may include an organic material that emits green light, and the third emission layer 370uv may include an organic material that emits blue light. The fourth emission layer 370uv may include an organic material that emits ultraviolet light. The emission layers 370r, 370g, 370b, and 370uv may include a low-molecular or high-molecular organic material. In FIG. 3, the emission layers 370r, 370g, 370b, and 370uv are shown as a single layer, but in an embodiment, auxiliary layers such as an electron injection layer, an electron transport layer, a hole transport layer, and a hole injection layer may be included above and below the emission layers 370r, 370g, 370b, and 370uv, and the hole injection layer and hole transport layer may be positioned under the emission layers 370r, 370g, 370b, and 370uv, while the electron transport layer and the electron injection layer may be positioned on the emission layers 370r, 370g, 370b, and 370uv.

The common electrode 270 may be positioned on the bank 350 and the emission layer 370. The common electrode 270 may be positioned entirely on the substrate 110. The common electrodes 270 positioned at each pixel PXr, PXg, PXb, and PXuv are connected to each other and may be integrated. The common electrode 270 is also called a cathode, and may be formed as a transparent conductive layer including Indium Tin Oxide (ITO), Indium Zinc Oxide (IZO), Indium Gallium Zinc Oxide (IGZO), and Indium Tin Zinc Oxide (ITZO). The common electrode 270 may have a translucent characteristic, and in this case, a micro-cavity may be formed together with the pixel electrode 191r, 191g, 191b, and 191uv. According to the micro-cavity structure, light with a specific wavelength may be emitted upwards by the spacing and characteristics between both electrodes.

In each pixel PXr, PXg, PXb, and PXuv, the pixel electrodes 191r, 191g, 191b, and 191uv, the emission layers 370r, 370g, 370b, and 370uv, and the common electrode 270 may configure the light-emitting elements EDr, EDg, EDb, and EDuv. In the first pixel PXr, the first pixel electrode 191r, the first emission layer 370r, and the common electrode 270 may constitute a first light-emitting element EDr, and red light may be emitted through the first pixel opening 351r. In the second pixel PXg, the second pixel electrode 191g, the second emission layer 370g, and the common electrode 270 may form a second light-emitting element EDg, and green light may be emitted through the second pixel opening 351g. In the third pixel PXb, the third pixel electrode 191b, the third emission layer 370b, and the common electrode 270 may constitute the third light-emitting element EDb, and blue light may be emitted through the third pixel opening 351b. In the fourth pixel PXuv, the fourth pixel electrode 191uv, the fourth emission layer 370uv, and the common electrode 270 may constitute the fourth light-emitting element EDuv, and ultraviolet light may be emitted through the fourth pixel opening 351uv. The size of the light emission area of the fourth light-emitting element EDuv may be smaller than the size of the light emission area of the first to third light-emitting elements EDr, EDg, and EDb in a plan view.

In the above, it has been described that the fourth light-emitting element EDuv of the fourth pixel PXuv includes the fourth emission layer 370uv capable of emitting ultraviolet light, but is not limited thereto. In various other ways, the fourth pixel PXuv may emit ultraviolet light, and the layer that emits ultraviolet light of the fourth pixel PXuv may be positioned on the same layer as or on a different layer from a layer that emits visible light of first to third pixel PXr, PXg, and PXb.

An encapsulation layer 400 may be disposed on the common electrode 270. The encapsulation layer 400 may include at least one inorganic layer and at least one organic layer. In the embodiment, the encapsulation layer 400 may include a first inorganic encapsulation layer 410, an organic encapsulation layer 420, and a second inorganic encapsulation layer 430. However, this is only an example, and the number of inorganic layers and organic layers constituting the encapsulation layer 400 may be variously changed. The encapsulation layer 400 is to protect light-emitting elements EDr, EDg, EDb, and EDuv from moisture or oxygen that may inflow from the outside, and one ends (or first ends) of each of the first inorganic encapsulation layer 410 and the second inorganic encapsulation layer 430 may be formed to be in direct contact each other.

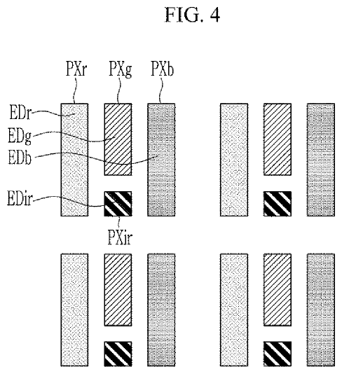

Next, the display device according to an embodiment is described with reference to FIG. 4.

The display device according to the embodiment shown in FIG. 4 may be substantially identical or similar to the display device according to the embodiment shown in FIG. 1 to FIG. 3, and a description of the same or similar parts is omitted. The embodiment differs from the previous embodiment at least in that the fourth light-emitting element emits infrared light, which will be further described below.

FIG. 4 is a schematic plan view of a display device according to an embodiment.

As shown in FIG. 4, a display device according to an embodiment includes pixels PXr, PXg, PXb, and PXir. Pixels PXr, PXg, PXb, and PXir may include a first pixel PXr, a second pixel PXg, a third pixel PXb, and a fourth pixel PXir. In each pixel PXr, PXg, PXb, and PXir, each light-emitting element EDr, EDg, EDb, and EDir may be positioned.

The first light-emitting element EDr, the second light-emitting element EDg, and the third light-emitting element EDb may emit visible light. The fourth light-emitting element EDir may emit infrared light. The fourth pixel PXir may be used for night photography or an infrared treatment.

The first to third pixels PXr, PXg, and PXb may be simultaneously driven. The fourth pixel PXir may be driven simultaneously with or separately from the first to third pixels PXr, PXg, and PXb. For example, the fourth pixel PXir may be selectively driven. In addition, the intensity of the infrared light emitted from the fourth pixel PXir may be adjusted with a button or app on the display device.

The first pixel PXr, the second pixel PXg, the third pixel PXb, and the fourth pixel PXir may form a pixel group, and pixel groups may be repeatedly disposed.

The size of the fourth pixel PXir may be relatively small compared to the size of the first to third pixels PXr, PXg, and PXb in a plan view. Accordingly, the size of the light emission area of the fourth light-emitting element EDir may be smaller than the size of the light emission area of the first to third light-emitting elements EDr, EDg, and EDb in a plan view. In the display device according to an embodiment, the aperture ratio may be improved by forming the fourth pixel PXir smaller than the first to third pixels PXr, PXg, and PXb. In the display device according to an embodiment, by forming the fourth pixel PXir relatively small, the influence of the infrared light emitted from the fourth pixel PXir on other pixels may be reduced.

The fourth pixel PXir may have a fourth emission layer including an organic material that emits infrared light. The fourth pixel electrode, the fourth emission layer, and the common electrode may constitute the fourth light-emitting element, and may emit infrared light through the fourth pixel opening. In this case, by making the size of the fourth pixel opening relatively small, the size of the fourth pixel PXir may be made relatively small, and the size of the light emission area of the fourth light-emitting element EDir may be relatively small.

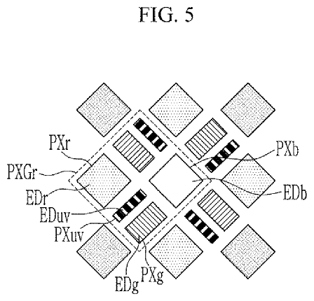

Next, the display device according to an embodiment is described with reference to FIG. 5.

The display device according to an embodiment shown in FIG. 5 may be substantially identical or similar to the display device according to the embodiment shown in FIG. 1 to FIG. 3, so the description for the same or similar parts is omitted. In the embodiment, the planar arrangement of the first to fourth pixels is different from the previous embodiment and will be further described below.

FIG. 5 is a schematic plan view of a display device according to an embodiment.

As shown in FIG. 5, the display device according to an embodiment includes pixels PXr, PXg, PXb, and PXuv. Each pixel PXr, PXg, PXb, and PXuv may include each light-emitting element EDr, EDg, EDb, and EDuv. The first to fourth light-emitting elements EDr, EDg, EDb, and EDuv may each emit light of different wavelengths. The first light-emitting element EDr, the second light-emitting element EDg, and the third light-emitting element EDb may emit visible light such as red, green, and blue. The fourth light-emitting element EDuv may emit light with a wavelength other than the visible light wavelength. For example, the fourth light-emitting element EDuv may emit ultraviolet light.

The first pixel PXr, the second pixel PXg, the third pixel PXb, and the fourth pixel PXuv may form one pixel group PXGr, and pixel groups may be repeatedly disposed. Pixels groups may be disposed in a matrix form along a row direction and a column direction. In one pixel group, one first pixel PXr, two second pixels PXg, one third pixel PXb, and two fourth pixels PXuv may be positioned. The pixel group PXGr may have an approximately diamond or rhombus shape. The first pixel PXr and the third pixel PXb may have a rhombus shape, and the second pixel PXg and the fourth pixel PXuv may have a rectangle shape. In the pixel group PXGr, the first pixel PXr and the third pixel PXb may be disposed on the left part and the right part, respectively. The second pixel PXg and the fourth pixel PXuv may be disposed on the upper part of the pixel group PXGr, and the second pixel PXg and the fourth pixel PXuv may be disposed on the right part of the pixel group PXGr. However, the shape and arrangement shape of the first to fourth pixels PXr, PXg, PXb, and PXuv are only an example, and may be variously changed.

The size of the fourth pixel PXuv may be relatively small compared to the size of the first to third pixels PXr, PXg, and PXb in a plan view. Accordingly, the size of the light emission area of the fourth light-emitting element EDuv may be smaller than the size of the light emission area of the first to third light-emitting elements EDr, EDg, and EDb in a plan view. In the display device according to an embodiment, the aperture ratio may be improved by forming the fourth pixel PXuv smaller than the first to third pixels PXr, PXg, and PXb. In the display device according to an embodiment, by forming the fourth pixel PXuv relatively small, the effect of ultraviolet light emitted from the fourth pixel PXuv on other pixels may be reduced.

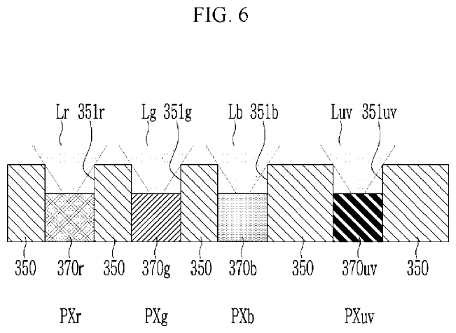

Next, the display device according to an embodiment is described with reference to FIG. 6.

The display device according to an embodiment shown in FIG. 6 may be substantially identical or similar to the display device according to the embodiment shown in FIG. 1 to FIG. 3, so the description for the same or similar parts is omitted. The embodiment differs from the previous embodiment at least in that the distance between the fourth pixel and other pixels is relatively far, and will be further described below.

FIG. 6 is a schematic cross-sectional view of a display device according to an embodiment. FIG. 6 shows only some layers of each pixel. A bank 350, pixel openings 351r, 351g, 351b, and 351uv, and emission layers 370r, 370g, 370b, and 370uv of each pixel PXr, PXg, PXb, and PXuv are shown.

As shown in FIG. 6, the display device according to an embodiment includes pixels PXr, PXg, PXb, and PXuv. The light-emitting element may be positioned in each pixel PXr, PXg, PXb, and PXuv. Each light-emitting element includes emission layers 370r, 370g, 370b, and 370uv, and the first to fourth emission layers 370r, 370g, 370b, and 370uv contain an organic material that emits light of different wavelengths, respectively. The first emission layer 370r, the second emission layer 370g, and the third emission layer 370b may include an organic material that emits visible light such as red, green, and blue. The fourth emission layer 370uv may include an organic material that emits light having a wavelength other than a visible light wavelength. For example, the fourth emission layer 370uv may include an organic material that emits ultraviolet light.

The emission layers 370r, 370g, 370b, and 370uv of each pixel PXr, PXg, PXb, and PXuv may be positioned within the pixel openings 351r, 351g, 351b, and 351uv of the bank 350. Accordingly, a bank 350 may be positioned between the emission layers 370r, 370g, 370b and 370uv, and the emission layers 370r, 370g, 370b, and 370uv may be partitioned by the bank 350, respectively. The bank 350 may be positioned between the first emission layer 370r and the second emission layer 370g, the bank 350 may be positioned between the second emission layer 370g and the third emission layer 370b, and the bank 350 may be positioned between the third emission layer 370b and the fourth emission layer 370uv.

In the previous embodiment, the width of the bank 350 may be constant, and in the embodiment, the width of the bank 350 may be variable. As the width of the bank 350 increases, the distance between the emission layers 370r, 370g, 370b, and 370uv positioned on both sides of the bank 350 may increase. For example, as the width of the bank 350 increases, the distance between the pixels PXr, PXg, PXb, and PXuv positioned on both sides of the bank 350 may increase. The width of the bank 350 positioned between the first emission layer 370r and the second emission layer 370g may be substantially equal to the width of the bank 350 positioned between the second emission layer 370g and the third emission layer 370b. The width of the bank 350 positioned between the third emission layer 370b and the fourth emission layer 370uv may be greater than the width of the bank 350 positioned between the second emission layer 370g and the third emission layer 370b. The width of the bank 350 surrounding the fourth emission layer 370uv may be greater than the width of the bank 350 positioned between the first emission layer 370r and the second emission layer 370g. The width of the bank 350 surrounding the fourth emission layer 370uv may be greater than the width of the bank 350 positioned between the second emission layer 370g and the third emission layer 370b. Therefore, the distance between the fourth emission layer 370uv and the first to third emission layers 370r, 370g, and 370b may be disposed relatively far compared to the distance between the first to third emission layers 370r, 370g, and 370b. For example, the distance between the fourth pixel PXuv and the first to third pixels PXr, PXg, and PXb may be disposed relatively far.

Light Lr, Lg, Lb, and Luv emitted from the emission layers 370r, 370g, 370b, and 370uv may diffuse to the surroundings. Thus, light Lr, Lg, Lb, and Luv emitted from each pixel PXr, PXg, PXb, and PXuv may affect other pixels PXr, PXg, PXb, and PXuv. The emission layers 370r, 370g, 370b, and 370uv may be damaged if the light (Luv) of the ultraviolet light emitted from the fourth pixel PXuv affects other pixels PXr, PXg, PXb, and PXuv. In the embodiment, by forming the distance between the fourth emission layer 370uv and the first to third emission layers 370r, 370g, and 370b relatively far, it may be prevented that ultraviolet light (Luv) from the fourth pixel PXuv affects other pixels.

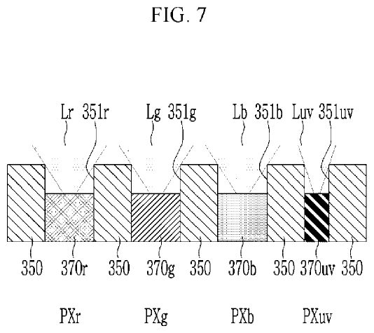

Next, a display device according to an embodiment is described with reference to FIG. 7.

The display device according to an embodiment shown in FIG. 7 may be substantially identical or similar to the display device according to the embodiment shown in FIG. 1 to FIG. 3, so the description for the same or similar parts is omitted. The embodiment differs from the previous embodiment at least in that the width of the fourth pixel is narrowed and is further described below.

FIG. 7 is a schematic cross-sectional view of a display device according to an embodiment.

As shown in FIG. 7, the display device according to an embodiment includes pixels PXr, PXg, PXb, and PXuv. The light-emitting element may be positioned in each pixel PXr, PXg, PXb, and PXuv. Each light-emitting element includes the emission layers 370r, 370g, 370b, and 370uv, and the first to fourth emission layers 370r, 370g, 370b, and 370uv may include the organic materials that emit light of different wavelengths, respectively. The first emission layer 370r, the second emission layer 370g, and the third emission layer 370b may include the organic materials that emit visible light such as red, green, and blue. The fourth emission layer 370uv may include an organic material that emits light having a wavelength other than a visible light wavelength. For example, the fourth emission layer 370uv may include an organic material that emits ultraviolet light.

In the previous embodiment, the width of each pixel PXr, PXg, PXb, and PXuv may be constant, and in the embodiment, the width of each pixel PXr, PXg, PXb, and PXuv may be different.

In the display device according to an embodiment, the width of the pixel openings 351r, 351g, 351b, and 351uv of the bank 350 may be different. The width of the fourth pixel opening 351uv may be relatively narrow compared to that of the first to third pixel openings 351r, 351g, and 351b. The width of the fourth emission layer 370uv may be narrower than that of the first to third emission layers 370r, 370g, and 370b. As the width of the pixel openings 351r, 351g, 351b, and 351uv is narrower, the range in which the light Lr, Lg, Lb, and Luv emitted from each pixel PXr, PXg, PXb, and PXuv is diffused may be reduced. The width of the fourth pixel opening 351uv is relatively narrow, and the diffusion range of the ultraviolet light emitted from the fourth emission layer 370uv may be relatively narrowed. Therefore, it is possible to prevent the light Luv emitted from the fourth pixel PXuv from affecting the first to third pixels PXr, PXg, and PXb.

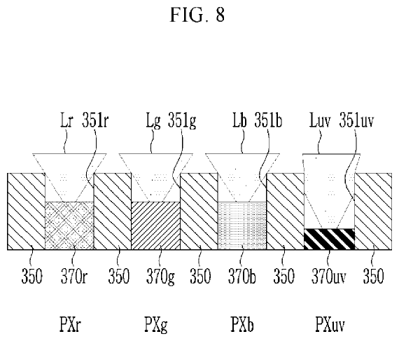

Next, a display device according to an embodiment is described with reference to FIG. 8.

The display device according to an embodiment shown in FIG. 8 may be substantially identical or similar to the display device according to the embodiment shown in FIG. 1 to FIG. 3, so the description for the same or similar parts is omitted. The embodiment differs from the previous embodiment at least in that the thickness of the fourth emission layer is formed thin and is further described below.

FIG. 8 is a schematic cross-sectional view of a display device according to an embodiment.

As shown in FIG. 8, the display device according to an embodiment includes pixels PXr, PXg, PXb, and PXuv. The light-emitting element may be positioned in each pixel PXr, PXg, PXb, and PXuv. Each light-emitting element include an emission layer (370r, 370g, 370b, and 370uv), and the first to fourth emission layers (370r, 370g, 370b, and 370uv) may each include an organic material that emits light of a different wavelength. The first emission layer 370r, the second emission layer 370g, and the third emission layer 370b may include an organic material that emits visible light such as red, green, and blue. The fourth emission layer 370uv may include an organic material that emits light having a wavelength other than a visible light wavelength. For example, the fourth emission layer 370uv may include an organic material that emits light of ultraviolet light.

In the previous embodiment, the thickness of the emission layers 370r, 370g, 370b, and 370uv of each pixel PXr, PXg, PXb, and PXuv may be constant, and in the embodiment, the thickness of the emission layers 370r, 370g, 370b, and 370uv of each pixel PXr, PXg, PXb, and PXuv may be different.

In the display device according to an embodiment, the thickness of the fourth emission layer 370uv may be relatively thinner than that of the first to third emission layers 370r, 370g, and 370b. As the thickness of the emission layers 370r, 370g, 370b, and 370uv is thinner, the range in which the light Lr, Lg, Lb, and Luv emitted from each pixel PXr, PXg, PXb, and PXuv is diffused may decrease. Since the thickness of the fourth emission layer 370uv is formed relatively thin, the diffusion range of the light Luv of the ultraviolet light emitted from the fourth emission layer 370uv may be relatively narrowed. Therefore, it is possible to prevent the light Luv emitted from the fourth pixel PXuv from affecting the first to third pixels PXr, PXg, and PXb.

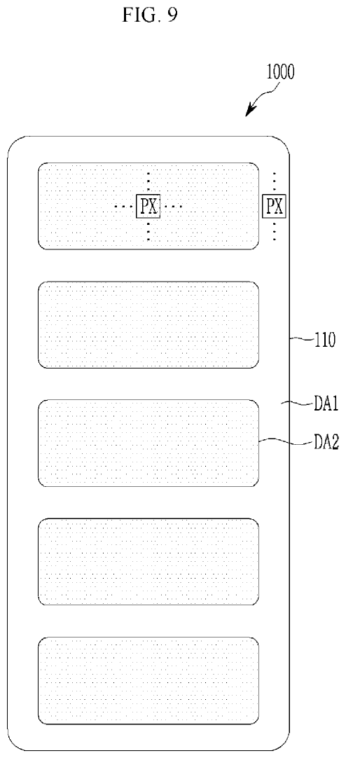

Next, a display device according to an embodiment is described with reference to FIG. 9 to FIG. 11.

The display devices according to an embodiment shown in FIG. 9 to FIG. 11 may be substantially identical or similar to the display device according to the embodiment shown in FIG. 1 to FIG. 3, so the description for the same or similar parts is omitted. The embodiment differs from the previous embodiment at least in that the fourth light-emitting element is positioned only in some regions and is further described below.

FIG. 9 to FIG. 11 are schematic plan views of a display device according to an embodiment.

As shown in FIG. 9 to FIG. 11, a display device 1000 according to an embodiment includes a substrate 110 and pixels PX disposed on the substrate 110.

The substrate 110 may include a first display area DA1 and a second display area DA2. The pixel PX positioned in the first display area DA1 may be composed of the pixels that emit visible light. For example, a first light-emitting element emitting red light, a second light-emitting element emitting green light, and a third light-emitting element emitting blue light may be positioned in the first display area DA1. The pixel PX positioned in the second display area DA2 may include the pixels that emit visible light and the pixels that emit invisible light. A wavelength of the invisible light may be shorter than about 400 nm or longer than about 700 nm. For example, in the second display area DA2, a first light-emitting element that emits red light, a second light-emitting element that emits green light, a third light-emitting element that emits blue light, and a fourth light-emitting element that emits ultraviolet light may be positioned. Thus, the light-emitting elements emitting visible light may be positioned in the first display area DA1 and the second display area DA2 of the substrate 110, and the fourth light-emitting element emitting ultraviolet light may be positioned only on the second display area DA2 of the substrate 110. For example, the light-emitting elements that emits visible light may be positioned entirely on the substrate 110, and the fourth light-emitting element that emits ultraviolet light may be positioned only in some regions on the substrate 110.

First, as shown in FIG. 9, the second display area DA2 may be formed as an approximate rectangle. The second display area DA2 may be disposed to be spaced apart with a constant interval from the upper side to the lower side of the substrate 110. The first display area DA1 may have a shape surrounding the second display area DA2.

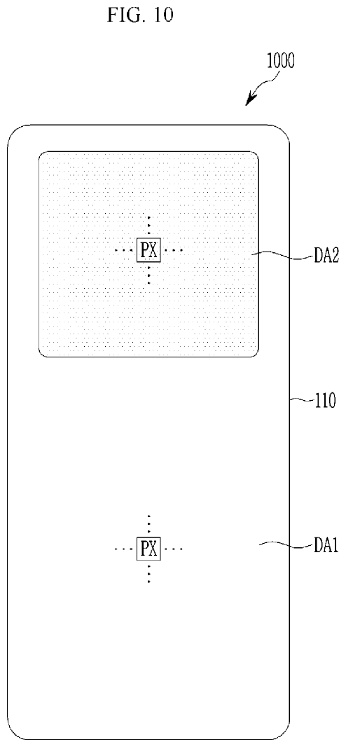

As shown in FIG. 10, the second display area DA2 may be formed as an approximate rectangle. The second display area DA2 may be positioned on the upper side of the substrate 110. The first display area DA1 may be positioned at the lower side of the substrate 110, and may have a shape surrounding the second display area DA2 at the upper side of the substrate 110.

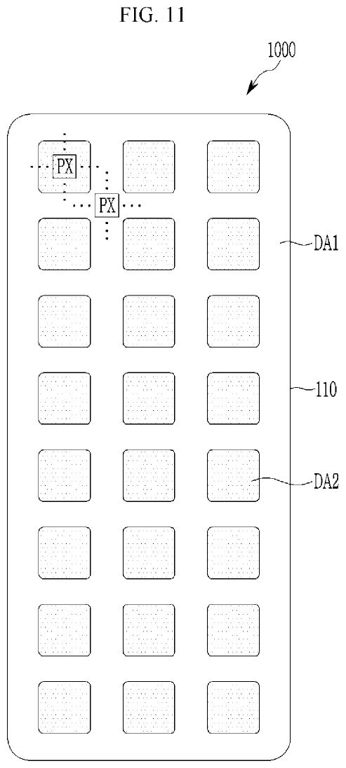

As shown in FIG. 11, the second display area DA2 may be formed as an approximately rectangle. The second display area DA2 may be disposed in a matrix form in the row direction and the column direction. The first display area DA1 may have a shape surrounding the second display area DA2.

The planar shape, size, number, and arrangement shape of the first display area DA1 and the second display area DA2 shown in FIG. 9 to FIG. 11 are only examples, and are not limited thereto. For example, the planar shape, size, number, and arrangement shape of the first display area DA1 and the second display area DA2 according to an embodiment may be variously changed.

In the display device according to an embodiment, as the fourth light-emitting element that emits ultraviolet light is positioned only in some regions, the aperture ratio may be improved and the effect of ultraviolet light from the fourth light-emitting element on other pixels may be reduced.

While this disclosure has been described in connection with what is presently considered to be practical embodiments, it is to be understood that the disclosure is not limited to the disclosed embodiments. On the contrary, it is intended to cover various modifications and equivalent arrangements included within the spirit and scope of the appended claims.

What is claimed is:

1. A display device comprising:
a substrate; and
a first light-emitting element, a second light-emitting element, a third light-emitting element, and a fourth light-emitting element disposed on the substrate, wherein
the first light-emitting element, the second light-emitting element, and the third light-emitting element emit visible light,
the fourth light-emitting element emits invisible light, and
a size of a light emission area of the fourth light-emitting element is smaller than a size of a light emission area of each of the first to third light-emitting elements in a plan view.

2. The display device of claim 1, wherein the fourth light-emitting element emits ultraviolet light or infrared light.

3. The display device of claim 1, further comprising:
a first pixel in which the first light-emitting element is disposed;
a second pixel in which the second light-emitting element is disposed;
a third pixel in which the third light-emitting element is disposed; and
a fourth pixel in which the fourth light-emitting element is disposed,
wherein the first pixel, the second pixel, the third pixel, and the fourth pixel form a pixel group.

4. The display device of claim 3, wherein
the first pixel, the second pixel, the third pixel, and the fourth pixel each have a rectangle shape, and
the second pixel and the fourth pixel are disposed between the first pixel and the third pixel.

5. The display device of claim 4, wherein
a width of each of the first pixel, the second pixel, the third pixel, and the fourth pixel in a row direction is equal to each other, and
a length of the fourth pixel in a column direction is shorter than a length of each of the first pixel, the second pixel, and the third pixel in a column direction.

6. The display device of claim 1, further comprising:
a first pixel in which the first light-emitting element is disposed;
a second pixel in which the second light-emitting element is disposed;
a third pixel in which the third light-emitting element is disposed; and
a fourth pixel in which the fourth light-emitting element is disposed,
wherein one first pixel, two second pixels, one third pixel, and two fourth pixels form a pixel group.

7. The display device of claim 6, wherein
the pixel group has a rhombus shape,
the first pixel and the third pixel each have a rhombus shape, and
the second pixel and the fourth pixel each have a rectangle shape.

8. The display device of claim 7, wherein
the first pixel is disposed on a left part of the pixel group,
the third pixel is disposed on a right part of the pixel group,
the second pixel and the fourth pixel are disposed on an upper part of the pixel group, and
the second pixel and the fourth pixel are disposed on a lower part of the pixel group.

9. The display device of claim 1, wherein
the first light-emitting element includes:
a first pixel electrode disposed on the substrate; and
a first emission layer disposed on the first pixel electrode and including an organic material emitting a first color light,
the second light-emitting element includes:
a second pixel electrode disposed on the substrate; and
a second emission layer disposed on the second pixel electrode and including an organic material emitting a second color light,
the third light-emitting element includes:
a third pixel electrode disposed on the substrate; and
a third emission layer disposed on the third pixel electrode and including an organic material emitting a third color light, and
the fourth light-emitting element includes:
a fourth pixel electrode disposed on the substrate; and
a fourth emission layer disposed on the fourth pixel electrode and including an organic material emitting ultraviolet light.

10. The display device of claim 9, wherein a distance between the fourth emission layer and the first to third emission layers is disposed farther than a distance between the first to third emission layers.

11. The display device of claim 9, further comprising
a bank including:
a first pixel opening overlapping the first pixel electrode;
a second pixel opening overlapping the second pixel electrode;
a third pixel opening overlapping the third pixel electrode; and
a fourth pixel opening overlapping the fourth pixel electrode, wherein
the first emission layer is disposed in the first pixel opening,
the second emission layer is disposed in the second pixel opening,
the third emission layer is disposed in the third pixel opening, and
the fourth emission layer is disposed in the fourth pixel opening.

12. The display device of claim 11, wherein
a width of the bank surrounding the fourth emission layer is greater than a width of the bank disposed between the first emission layer and the second emission layer, and
a width of the bank surrounding the fourth emission layer is greater than a width of the bank disposed between the second emission layer and the third emission layer.

13. The display device of claim 9, wherein a width of the fourth pixel opening is narrower than a width of each of the first to third pixel opening.

14. The display device of claim 13, wherein a width of the fourth emission layer is narrower than a width of each of the first to third emission layers.

15. The display device of claim 9, wherein a thickness of the fourth emission layer is thinner than a thickness of each of the first to third emission layers.

16. The display device of claim 9, further comprising:
a common electrode disposed on the first emission layer, the second emission layer, the third emission layer, and the fourth emission layer and extending from the first emission layer to the fourth emission layer.

17. The display device of claim 1, wherein
the substrate includes a first display area and a second display area, and
the fourth light-emitting element is not disposed in the first display area and is disposed in the second display area.

18. The display device of claim 17, wherein
the first light-emitting element, the second light-emitting element, and the third light-emitting element are disposed in the first display area, and
the first light-emitting element, the second light-emitting element, the third light-emitting element, and the fourth light-emitting element are disposed in the second display area.

19. The display device of claim 17, wherein
the substrate includes second display areas, and
second display areas are spaced apart by a constant interval.

20. The display device of claim 17, wherein
the substrate includes second display areas, and
second display areas are disposed in a row direction and a column direction.
